(19) United States
(12) Patent Application Publication
(10) Pub. No.: US 2025/0223563 A1
(43) Pub. Date: Jul. 10, 2025

(54) Canine Universal Donor Mesenchymal Stem Cells

(71) Applicant: CREATIVE MEDICAL TECHNOLOGIES, INC., Phoenix, AZ (US)

(72) Inventors: Thomas Ichim, San Diego, CA (US); Timothy Warbington, Phoenix, AZ (US)

(73) Assignee: CREATIVE MEDICAL TECHNOLOGIES, INC., Phoenix, AZ (US)

(21) Appl. No.: 19/015,393

(22) Filed: Jan. 9, 2025

Related U.S. Application Data
(60) Provisional application No. 63/619,235, filed on Jan. 9, 2024.

(51) Int. Cl. C12N 5/0775; A61K 35/28

(52) U.S. Cl. CPC C12N 5/0668; A61K 35/28

(57) ABSTRACT

Novel populations of canine derived stem cells useful for treatment across allogeneic barriers. The stem cells can be of mesenchymal lineage optimized to possess enhanced proliferative activity in vitro and in vivo without significant loss of therapeutic functionality. Said therapeutic activities include angiogenesis, stimulation of growth factor production, antifibrotic effects, neurogenesis, immune modulation, and antiapoptotic effects.

### CROSS-REFERENCE TO RELATED APPLICATIONS

The present application claims benefit of U.S. Provisional Patent Application Ser. No. 63/619,235, filed on Jan. 9, 2024 entitled CANINE UNIVERSAL DONOR MESENCHYMAL STEM CELLS, the contents of which are incorporated herein by reference in its entirety.

### FIELD OF THE INVENTION

The invention relates to the field of veterinary medicine, more specifically, the invention pertains to the field of canine regenerative medicine, more specifically, the invention relates to the utilization of induced pluripotent stem cell (iPSC) technology to generate consistent, therapeutically potent cells of the mesenchymal stem cell lineage.

### BACKGROUND OF THE INVENTION

Mesenchymal stem cells, or mesenchymal stromal cells (MSC) are believed to provide structural support in different organs and regulate the flow of some substances. The stromal origin is characterized by their quick adhesion in culture surface as well as their fibroblastic-like morphology. In addition, they present a high and fast proliferation in simple and accessible culture medium and can be maintained in vitro without karyotype alterations for several passages.

Unfortunately, passaging of canine MSC appears more limited in vitro as compared to human or mouse MSC. The current invention aims to overcome this limitation by deriving “younger” MSC through the use of pluripotent stem cell sources for “de novo” differentiation.

Classically, it is known that MSC have the ability to differentiate into several cell types such as adipocytes, osteocytes, and chondrocytes, from the mesodermal germ layer. This plasticity depends on the extra-cellular matrix environment and soluble growth factors. Some authors could induce the differentiation of MSC in cells of other embryonic germ layers, such as neurons which are originated in ectoderm, and hepatocytes, derived from endoderm.

Due to their plasticity, the MSC are considered the most important cell type for regenerative medicine, and are the most widely studied in preclinical and clinical trials. Their advantages for clinical application include the easy isolation and high yield, high plasticity, and the ability to mediate inflammation and to promote cell growth, cell differentiation, and tissue repair by immunomodulation and immunosuppression, and are exempt from ethical implications.

To date, in human medicine, MSC derived from bone marrow have been the most intensively studied; however, invasive procedures are required for their isolation and the quantity and quality of isolated cells vary according to the donor age. Low frequencies of MSC are found in bone marrow aspirates compared to the total cells compounding the bone marrow stroma.

The invention provides generation of canine MSC, in part, from pluripotent stem cell sources in order to provide cells of a higher “potency” as well as larger proliferative/expansion potential.

### SUMMARY OF THE INVENTION

A summary of the invention is provided below with respect to the following numbered aspects.

1. A canine derived mesenchymal stem cell derived from an induced pluripotent stem cell.

2. The canine derived mesenchymal stem cell of aspect 1, wherein said stem cell is capable of suppressing T cell proliferation.

3. The canine derived mesenchymal stem cell of aspect 2, wherein said stem cell is capable of enhancing T regulatory cell proliferation.

4. The canine derived mesenchymal stem cell of aspect 2, wherein said stem cell is capable of enhancing angiogenesis.

5. The canine derived mesenchymal stem cell of aspect 2, wherein said stem cell is capable of enhancing neurogenesis.

6. The canine derived mesenchymal stem cell of aspect 2, wherein said stem cell is capable of enhancing tissue regeneration.

7. The canine derived mesenchymal stem cell of aspect 2, wherein said stem cell is capable of enhancing antiapoptotic activities.

8. The canine derived mesenchymal stem cell of aspect 1, wherein said cell is capable of more than 20 doublings without losing therapeutic activity by more than 10%.

9. The canine derived mesenchymal stem cell of aspect 8, wherein said therapeutic activity is ability of conditioned media from said canine derived mesenchymal stem cell to stimulate proliferation of canine derived umbilical endothelial cells by more than 25% as compared to baseline proliferation.

10. The canine derived mesenchymal stem cell of aspect 8, wherein said therapeutic activity is ability of conditioned media from said canine derived mesenchymal stem cell to stimulate production of canine FGF-1 from canine derived umbilical endothelial cells by more than 25% as compared to baseline proliferation.

11. The canine derived mesenchymal stem cell of aspect 8, wherein said therapeutic activity is ability of conditioned media from said canine derived mesenchymal stem cell to stimulate production of canine FGF-2 from canine derived umbilical endothelial cells by more than 25% as compared to baseline proliferation.

12. The canine derived mesenchymal stem cell of aspect 8, wherein said therapeutic activity is ability of conditioned media from said canine derived mesenchymal stem cell to stimulate production of canine VEGF from canine derived umbilical endothelial cells by more than 25% as compared to baseline proliferation.

13. The canine derived mesenchymal stem cell of aspect 1, wherein said induced pluripotent stem cells are cultured under conditions conducive to form embryoid bodies (EBs), wherein subsequently said EBs are exposed to a mesenchyme-specific medium containing 1.25(OH)2D3 to promote formation of EBs having cells of the mesenchymal lineage.

14. The canine derived mesenchymal stem cell of aspect 13, wherein said mesenchyme-specific media contains VEGF.

15. The canine derived mesenchymal stem cell of aspect 13, wherein said mesenchyme-specific media contains BMP2.

16. The canine derived mesenchymal stem cell of aspect 13, wherein said mesenchyme-specific media contains BMP3.

17. The canine derived mesenchymal stem cell of aspect 13, wherein said mesenchyme-specific media contains BMP4.

18. The canine derived mesenchymal stem cell of aspect 13, wherein said mesenchyme-specific media contains ascorbic acid.

19. The canine derived mesenchymal stem cell of aspect 13, wherein said mesenchyme-specific media contains dexamethasone.

20. The canine derived mesenchymal stem cell of aspect 13, wherein said mesenchyme-specific media contains retinoic acid.

21. The canine derived mesenchymal stem cell of aspect 13, wherein said mesenchyme-specific media contains 1.25(OH)2D3.

22. The canine derived mesenchymal stem cell of aspect 13, wherein said mesenchyme-specific media contains β-glycerophosphate.

23. The canine derived mesenchymal stem cell of aspect 13, wherein said mesenchyme-specific media contains canine monocyte conditioned media.

24. The canine derived mesenchymal stem cell of aspect 23, wherein said canine monocyte conditioned media is generated by culturing canine peripheral blood mononuclear cells in an adherent vessel, allowing monocytes to adhere to said vessel, washing off non-adherent cells from said vessel, culturing said monocytes in a liquid media, and subsequently collecting said liquid media as a conditioned media.

25. The canine derived mesenchymal stem cell of aspect 24, wherein said monocytes are first positively selected for expression of canine CD14 prior to allowing to adhere.

26. The canine derived mesenchymal stem cell of aspect 24, wherein said monocytes are first positively selected for expression of canine CD16 prior to allowing to adhere.

27. The canine derived mesenchymal stem cell of aspect 24, wherein said monocytes are first positively selected for expression of canine CD24 prior to allowing to adhere.

28. The canine derived mesenchymal stem cell of aspect 24, wherein said monocytes are first positively selected for expression of canine CD38 prior to allowing to adhere.

29. The canine derived mesenchymal stem cell of aspect 24, wherein said monocytes are first positively selected for expression of canine CD86 prior to allowing to adhere.

30. The canine derived mesenchymal stem cell of aspect 24, wherein said monocytes are first positively selected for expression of canine CD107b prior to allowing to adhere.

31. The canine derived mesenchymal stem cell of aspect 24, wherein said monocytes are stimulated with a priming agent to increase production of mesenchymal stem cell growth factors.

32. The canine derived mesenchymal stem cell of aspect 31, wherein said priming agent is interferon gamma.

33. The canine derived mesenchymal stem cell of aspect 32, wherein said interferon gamma is added at a concentration and duration sufficient to increase production of canine IL-1 beta from said monocytes by at least 10% compared to baseline.

34. The canine derived mesenchymal stem cell of aspect 31, wherein said interferon gamma is added at a concentration and duration sufficient to increase production of canine IL-1 beta from said monocytes by at least 25% compared to baseline.

35. The canine derived mesenchymal stem cell of aspect 31, wherein said interferon gamma is added at a concentration and duration sufficient to increase production of canine IL-1 beta from said monocytes by at least 100% compared to baseline.

36. The canine derived mesenchymal stem cell of aspect 31, wherein said priming agent is beta glucan.

37. The canine derived mesenchymal stem cell of aspect 36, wherein said beta glucan is added at a concentration and duration sufficient to increase production of canine IL-1 beta from said monocytes by at least 10% compared to baseline.

38. The canine derived mesenchymal stem cell of aspect 36, wherein said beta glucan is added at a concentration and duration sufficient to increase production of canine IL-1 beta from said monocytes by at least 25% compared to baseline.

39. The canine derived mesenchymal stem cell of aspect 36, wherein said beta glucan is added at a concentration and duration sufficient to increase production of canine IL-1 beta from said monocytes by at least 100% compared to baseline.

40. The canine derived mesenchymal stem cell of aspect 31, wherein said priming agent is lipopolysaccharide.

41. The canine derived mesenchymal stem cell of aspect 40, wherein said lipopolysaccharide is added at a concentration and duration sufficient to increase production of canine IL-1 beta from said monocytes by at least 10% compared to baseline.

42. The canine derived mesenchymal stem cell of aspect 40, wherein said lipopolysaccharide is added at a concentration and duration sufficient to increase production of canine IL-1 beta from said monocytes by at least 25% compared to baseline.

43. The canine derived mesenchymal stem cell of aspect 40, wherein said lipopolysaccharide is added at a concentration and duration sufficient to increase production of canine IL-1 beta from said monocytes by at least 100% compared to baseline.

44. The canine derived mesenchymal stem cell of aspect 31, wherein said priming agent is imiquimod.

45. The canine derived mesenchymal stem cell of aspect 44, wherein said imiquimod is added at a concentration and duration sufficient to increase production of canine IL-1 beta from said monocytes by at least 10% compared to baseline.

46. The canine derived mesenchymal stem cell of aspect 44, wherein said imiquimod is added at a concentration and duration sufficient to increase production of canine IL-1 beta from said monocytes by at least 25% compared to baseline.

47. The canine derived mesenchymal stem cell of aspect 44, wherein said imiquimod is added at a concentration and duration sufficient to increase production of canine IL-1 beta from said monocytes by at least 100% compared to baseline.

48. The canine derived mesenchymal stem cell of aspect 31, wherein said priming agent is flagellin.

49. The canine derived mesenchymal stem cell of aspect 48, wherein said flagellin is added at a concentration and duration sufficient to increase production of canine IL-1 beta from said monocytes by at least 10% compared to baseline.

50. The canine derived mesenchymal stem cell of aspect 48, wherein said flagellin is added at a concentration and duration sufficient to increase production of canine IL-1 beta from said monocytes by at least 25% compared to baseline.

51. The canine derived mesenchymal stem cell of aspect 48, wherein said flagellin is added at a concentration and duration sufficient to increase production of canine IL-1 beta from said monocytes by at least 100% compared to baseline.

52. The canine derived mesenchymal stem cell of aspect 31, wherein said priming agent is TNF-alpha.

53. The canine derived mesenchymal stem cell of aspect 52, wherein said TNF-alpha is added at a concentration and duration sufficient to increase production of canine IL-1 beta from said monocytes by at least 10% compared to baseline.

54. The canine derived mesenchymal stem cell of aspect 52, wherein said TNF-alpha is added at a concentration and duration sufficient to increase production of canine IL-1 beta from said monocytes by at least 25% compared to baseline.

55. The canine derived mesenchymal stem cell of aspect 52, wherein said TNF-alpha is added at a concentration and duration sufficient to increase production of canine IL-1 beta from said monocytes by at least 100% compared to baseline.

56. The canine derived mesenchymal stem cell of aspect 31, wherein said priming agent is interleukin-6.

57. The canine derived mesenchymal stem cell of aspect 56, wherein said interleukin-6 is added at a concentration and duration sufficient to increase production of canine IL-1 beta from said monocytes by at least 10% compared to baseline.

58. The canine derived mesenchymal stem cell of aspect 56, wherein said interleukin-6 is added at a concentration and duration sufficient to increase production of canine IL-1 beta from said monocytes by at least 25% compared to baseline.

59. The canine derived mesenchymal stem cell of aspect 56, wherein said interleukin-6 is added at a concentration and duration sufficient to increase production of canine IL-1 beta from said monocytes by at least 100% compared to baseline.

60. The canine derived mesenchymal stem cell of aspect 31, wherein said priming agent is interleukin-8.

61. The canine derived mesenchymal stem cell of aspect 60, wherein said interleukin-8 is added at a concentration and duration sufficient to increase production of canine IL-1 beta from said monocytes by at least 10% compared to baseline.

62. The canine derived mesenchymal stem cell of aspect 60, wherein said interleukin-8 is added at a concentration and duration sufficient to increase production of canine IL-1 beta from said monocytes by at least 25% compared to baseline.

63. The canine derived mesenchymal stem cell of aspect 60, wherein said interleukin-8 is added at a concentration and duration sufficient to increase production of canine IL-1 beta from said monocytes by at least 100% compared to baseline.

64. The canine derived mesenchymal stem cell of aspect 31, wherein said priming agent is interleukin-12.

65. The canine derived mesenchymal stem cell of aspect 64, wherein said interleukin-12 is added at a concentration and duration sufficient to increase production of canine IL-1 beta from said monocytes by at least 10% compared to baseline.

66. The canine derived mesenchymal stem cell of aspect 64, wherein said interleukin-12 is added at a concentration and duration sufficient to increase production of canine IL-1 beta from said monocytes by at least 25% compared to baseline.

67. The canine derived mesenchymal stem cell of aspect 64, wherein said interleukin-12 is added at a concentration and duration sufficient to increase production of canine IL-1 beta from said monocytes by at least 100% compared to baseline.

68. The canine derived mesenchymal stem cell of aspect 31, wherein said priming agent is interleukin-15.

69. The canine derived mesenchymal stem cell of aspect 68, wherein said interleukin-15 is added at a concentration and duration sufficient to increase production of canine IL-1 beta from said monocytes by at least 10% compared to baseline.

70. The canine derived mesenchymal stem cell of aspect 68, wherein said interleukin-15 is added at a concentration and duration sufficient to increase production of canine IL-1 beta from said monocytes by at least 25% compared to baseline.

71. The canine derived mesenchymal stem cell of aspect 68, wherein said interleukin-15 is added at a concentration and duration sufficient to increase production of canine IL-1 beta from said monocytes by at least 100% compared to baseline.

72. The canine derived mesenchymal stem cell of aspect 31, wherein said priming agent is interleukin-18.

73. The canine derived mesenchymal stem cell of aspect 72, wherein said interleukin-18 is added at a concentration and duration sufficient to increase production of canine IL-1 beta from said monocytes by at least 10% compared to baseline.

74. The canine derived mesenchymal stem cell of aspect 72, wherein said interleukin-18 is added at a concentration and duration sufficient to increase production of canine IL-1 beta from said monocytes by at least 25% compared to baseline.

75. The canine derived mesenchymal stem cell of aspect 72, wherein said interleukin-18 is added at a concentration and duration sufficient to increase production of canine IL-1 beta from said monocytes by at least 100% compared to baseline.

76. The canine derived mesenchymal stem cell of aspect 1, wherein said mesenchymal stem cell expresses the Cba-1 gene.

77. The canine derived mesenchymal stem cell of aspect 1, wherein said mesenchymal stem cell expresses the Msx2 gene.

78. The canine derived mesenchymal stem cell of aspect 1, wherein said mesenchymal stem cell expresses the D1x5 gene.

79. The canine derived mesenchymal stem cell of aspect 1, wherein said mesenchymal stem cell is generated from said iPSC in a manner to express higher amounts of hTERT as compared to a mesenchymal stem cell isolated from canine tissue.

80. The canine mesenchymal stem cell of aspect 79, wherein said canine tissue is amniotic membrane.

81. The canine mesenchymal stem cell of aspect 79, wherein said canine tissue is amniotic fluid.

82. The canine mesenchymal stem cell of aspect 79, wherein said canine tissue is adipose tissue.

83. The canine mesenchymal stem cell of aspect 79, wherein said canine tissue is dental pulp tissue.

84. The canine mesenchymal stem cell of aspect 79, wherein said canine tissue is bone marrow.

85. The canine mesenchymal stem cell of aspect 79, wherein said canine tissue is thymic tissue.

86. The canine mesenchymal stem cell of aspect 79, wherein said canine tissue is umbilical cord blood.

87. The canine mesenchymal stem cell of aspect 79, wherein said canine tissue is umbilical cord tissue.

88. The canine mesenchymal stem cell of aspect 79, wherein said canine tissue is omental tissue.

89. The canine mesenchymal stem cell of aspect 79, wherein said canine tissue is deciduous tooth tissue.

90. The canine derived mesenchymal stem cell of aspect 1, wherein said mesenchymal stem cell is generated from said iPSC in a manner to express higher amounts of c-kit as compared to a mesenchymal stem cell isolated from canine tissue.

91. The canine derived mesenchymal stem cell of aspect 1, wherein said mesenchymal stem cell is generated from said iPSC in a manner to express higher amounts of CD73 as compared to a mesenchymal stem cell isolated from canine tissue.

91. The canine derived mesenchymal stem cell of aspect 1, wherein said mesenchymal stem cell is generated from said iPSC in a manner to express higher amounts of c-met as compared to a mesenchymal stem cell isolated from canine tissue.

92. The canine derived mesenchymal stem cell of aspect 1, wherein said mesenchymal stem cell is generated from said iPSC in a manner to express higher amounts of CD105 as compared to a mesenchymal stem cell isolated from canine tissue.

93. The canine derived mesenchymal stem cell of aspect 1, wherein said mesenchymal stem cell is generated from said iPSC in a manner to express higher amounts of soluble HLA-G as compared to a mesenchymal stem cell isolated from canine tissue.

94. The canine derived mesenchymal stem cell of aspect 1, wherein said mesenchymal stem cell is generated from said iPSC in a manner to express higher amounts of soluble TNF-alpha receptor as compared to a mesenchymal stem cell isolated from canine tissue.

95. The canine derived mesenchymal stem cell of aspect 1, wherein said mesenchymal stem cell is generated from said iPSC in a manner to express higher amounts of interleukin-10 as compared to a mesenchymal stem cell isolated from canine tissue.

96. The canine derived mesenchymal stem cell of aspect 1, wherein said mesenchymal stem cell is generated from said iPSC in a manner to express higher amounts of soluble Fas ligand as compared to a mesenchymal stem cell isolated from canine tissue.

97. The canine derived mesenchymal stem cell of aspect 1, wherein said mesenchymal stem cell is generated from said iPSC in a manner to express higher amounts of interleukin-20 as compared to a mesenchymal stem cell isolated from canine tissue.

98. The canine derived mesenchymal stem cell of aspect 1, wherein said mesenchymal stem cell is generated from said iPSC in a manner to express higher amounts of interleukin-35 as compared to a mesenchymal stem cell isolated from canine tissue.

99. The canine derived mesenchymal stem cell of aspect 1, wherein said mesenchymal stem cell is generated from said iPSC in a manner to express higher amounts of interleukin-37 as compared to a mesenchymal stem cell isolated from canine tissue.

100. The canine derived mesenchymal stem cell of aspect 1, wherein said mesenchymal stem cell is generated from said iPSC in a manner to express higher amounts of interleukin-38 as compared to a mesenchymal stem cell isolated from canine tissue.

101. The canine derived mesenchymal stem cell of aspect 1, wherein said mesenchymal stem cell is generated from said iPSC in a manner to express higher amounts of EGF as compared to a mesenchymal stem cell isolated from canine tissue.

102. The canine derived mesenchymal stem cell of aspect 1, wherein said mesenchymal stem cell is generated from said iPSC in a manner to express higher amounts of FGF-1 as compared to a mesenchymal stem cell isolated from canine tissue.

103. The canine derived mesenchymal stem cell of aspect 1, wherein said mesenchymal stem cell is generated from said iPSC in a manner to express higher amounts of angiopoietin as compared to a mesenchymal stem cell isolated from canine tissue.

104. The canine derived mesenchymal stem cell of aspect 1, wherein said mesenchymal stem cell is generated from said iPSC in a manner to express higher amounts of FGF-2 as compared to a mesenchymal stem cell isolated from canine tissue.

105. A method of treating canine hepatic failure comprising the steps of: a) obtaining a canine induced pluripotent stem cell; b) creating embryoid bodies from said canine derived induced pluripotent stem cells; c) isolating mesenchymal progenitor cells from said embryoid bodies; d) expanding mesenchymal stem cell populations from said embryoid bodies; e) priming said mesenchymal stem cells towards hepatic differentiation; and f) administering said hepatic primed mesenchymal stem cells in a canine in need of therapy.

106. The method of aspect 105, wherein said canine induced pluripotent cell is generated by transfection with dedifferentiation factors.

107. The method of aspect 106, wherein one or more histone deacetylase inhibitors are added to said dedifferentiation factors.

108. The method of aspect 106, wherein said cells are cultured under conditions of hypoxia.

109. The method of aspect 108, wherein said conditions of hypoxia are sufficient to induce translocation of HIF-1 alpha by more than 10% as compared to baseline.

110. The method of aspect 108, wherein said conditions of hypoxia are sufficient to induce translocation of HIF-1 alpha by more than 25% as compared to baseline.

111. The method of aspect 108, wherein said conditions of hypoxia are sufficient to induce translocation of HIF-1 alpha by more than 100% as compared to baseline.

112. The method of aspect 108, wherein said cells are cultured under conditions of cellular stress.

113. The method of aspect 108, wherein said condition of cellular stress is hyperthermia.

114. The method of aspect 108, wherein said condition of cellular stress is hypertonic stress.

115. The method of aspect 108, wherein said condition of cellular stress is hypotonic stress.

116. The method of aspect 105, wherein said differentiation of said embryoid body derived cells into said mesenchymal stem cells is performed by culture in VEGF.

117. The method of aspect 105, wherein said differentiation of said embryoid body derived cells into said mesenchymal stem cells is performed by culture in BMP1.

118. The method of aspect 105, wherein said differentiation of said embryoid body derived cells into said mesenchymal stem cells is performed by culture in BMP1 and VEGF.

119. The method of aspect 105, wherein said differentiation of said embryoid body derived cells into said mesenchymal stem cells is performed by culture in BMP2.

120. The method of aspect 105, wherein said differentiation of said embryoid body derived cells into said mesenchymal stem cells is performed by culture in BMP2 and VEGF.

121. The method of aspect 105, wherein said differentiation of said embryoid body derived cells into said mesenchymal stem cells is performed by culture in BMP3.

123. The method of aspect 105, wherein said differentiation of said embryoid body derived cells into said mesenchymal stem cells is performed by culture in BMP3 and VEGF.

124. The method of aspect 105, wherein said differentiation of said embryoid body derived cells into said mesenchymal stem cells is performed by culture in BMP4.

125. The method of aspect 105, wherein said differentiation of said embryoid body derived cells into said mesenchymal stem cells is performed by culture in BMP4 and VEGF.

126. The method of aspect 105, wherein said differentiation of said embryoid body derived cells into said mesenchymal stem cells is performed by culture in BMP4 and monocyte conditioned media.

127. The method of aspect 105, wherein said differentiation of said embryoid body derived cells into said mesenchymal stem cells is performed by culture in BMP4, VEGF and monocyte conditioned media.

128. The method of aspect 105, wherein said differentiation of said embryoid body derived cells into said mesenchymal stem cells is performed by selection of naturally differentiated mesenchymal stem cell progenitors residing in the embryoid body.

129. The method of aspect 128, wherein said selection of said naturally differentiated mesenchymal stem cell progenitors is performed using positive selection for STRO-1.

130. The method of aspect 128, wherein said selection of said naturally differentiated mesenchymal stem cell progenitors is performed using positive selection for STRO-3.

131. The method of aspect 128, wherein said selection of said naturally differentiated mesenchymal stem cell progenitors is performed using positive selection for STRO-4.

132. The method of aspect 128, wherein said selection of said naturally differentiated mesenchymal stem cell progenitors is performed using positive selection for c-kit.

133. The method of aspect 128, wherein said selection of said naturally differentiated mesenchymal stem cell progenitors is performed using positive selection for CD90.

134. The method of aspect 128, wherein said selection of said naturally differentiated mesenchymal stem cell progenitors is performed using positive selection for CD73.

135. The method of aspect 128, wherein said selection of said naturally differentiated mesenchymal stem cell progenitors is performed using positive selection for CD105.

136. The method of aspect 128, wherein said selection of said naturally differentiated mesenchymal stem cell progenitors is performed using positive selection for LIF receptor.

137. The method of aspect 128, wherein said selection of said naturally differentiated mesenchymal stem cell progenitors is performed using positive selection for endoglin receptor.

138. The method of aspect 128, wherein said selection of said naturally differentiated mesenchymal stem cell progenitors is performed using positive selection for activin receptor.

139. The method of aspect 128, wherein said selection of said naturally differentiated mesenchymal stem cell progenitors is performed using positive selection for TGF-beta receptor.

140. The method of aspect 128, wherein said selection of said naturally differentiated mesenchymal stem cell progenitors is performed using positive selection for NGF receptor.

141. The method of aspect 128, wherein said selection of said naturally differentiated mesenchymal stem cell progenitors is performed by selecting for cells possessing ability to efflux rhodamine 231.

142. The method of aspect 128, wherein said selection of said naturally differentiated mesenchymal stem cell progenitors is performed by selecting for cells expressing ABC drug transporter protein.

143. The method of aspect 105, wherein said hepatic differentiation is achieved by culturing said mesenchymal stem cells in the presence of hepatocyte growth factor.

144. The method of aspect 143, wherein said hepatocyte growth factor is added at a concentration and time duration sufficient to induce an increase of 10% or more in expression of the albumin gene.

145. The method of aspect 143, wherein said hepatocyte growth factor is added at a concentration and time duration sufficient to induce an increase of 20% or more in expression of the albumin gene.

146. The method of aspect 143, wherein said hepatocyte growth factor is added at a concentration and time duration sufficient to induce an increase of 100% or more in expression of the albumin gene.

147. The method of aspect 143, wherein said hepatocyte growth factor is added at a concentration and time duration sufficient to induce an increase of 200% or more in expression of the albumin gene.

148. The method of aspect 143, wherein said hepatocyte grow factor is added to said mesenchymal stem cells after addition of a histone deacetylase inhibitor.

149. The method of aspect 148, wherein said histone deacetylase inhibitor is added at a concentration and time duration to increase expression of CD73 on said mesenchymal stem cells by 20%.

150. The method of aspect 148, wherein said histone deacetylase inhibitor is added at a concentration and time duration to increase expression of CD73 on said mesenchymal stem cells by 100%.

151. The method of aspect 148, wherein said histone deacetylase inhibitor is added at a concentration and time duration to increase expression of CD73 on said mesenchymal stem cells by 200%.

152. The method of aspect 148, wherein said histone deacetylase inhibitor is trichostatin A.

153. The method of aspect 148, wherein said histone deacetylase inhibitor is phenylbutyrate.

154. The method of aspect 148, wherein said histone deacetylase inhibitor is valproic acid.

155. The method of aspect 148, wherein said histone deacetylase inhibitor is vorinostat.

156. The method of aspect 148, wherein said histone deacetylase inhibitor is romidepsin.

157. The method of aspect 148, wherein said histone deacetylase inhibitor is panobinostat.

158. The method of aspect 148, wherein said histone deacetylase inhibitor is belinostat.

159. The method of aspect 148, wherein said histone deacetylase inhibitor is a molecule capable of inducing RNA interference to one or more histone deacetylases.

160. The method of aspect 159, wherein said molecule capable of inducing RNA interference is a short hairpin RNA.

161. The method of aspect 159, wherein said molecule capable of inducing RNA interference is a short interfering RNA.

162. The method of aspect 148, wherein said histone deacetylase inhibitor is a morpholino targeting one or more histone deacetylases.

163. The method of aspect 148, wherein said histone deacetylase inhibitor is an aptamer targeting one or more histone deacetylases.

164. The method of aspect 148, wherein said histone deacetylase inhibitor is a somomer targeting one or more histone deacetylases.

165. The method of aspect 148, wherein said histone deacetylase inhibitor is a ribozyme targeting one or more histone deacetylases.

166. The method of aspect 148, wherein said histone deacetylase inhibitor is a hammerhead ribozyme targeting one or more histone deacetylases.

167. The method of aspect 148, wherein said histone deacetylase inhibitor is an antisense oligonucleotide targeting one or more histone deacetylases.

168. The method of aspect 105, wherein said generated hepatocyte primed mesenchymal stem cells possess an enhanced proclivity for in vivo liver differentiation.

169. The method of aspect 168, wherein said enhanced proclivity for liver differentiation is accomplished by transfection of hepatocyte growth factor.

170. The method of aspect 168, wherein said enhanced proclivity for liver differentiation is accomplished by pretreatment with hepatocyte growth factor.

171. The method of aspect 168, wherein said enhanced proclivity for liver differentiation is accomplished by transfection with CXCR4.

172. The method of aspect 168, wherein said enhanced proclivity for liver differentiation is accomplished by insertion of one or more molecules capable of directing homing of hepatic primed cells towards the hepatic microenvironment.

173. The method of aspect 172, wherein said molecule capable of directing homing to the liver microenvironment are hepatic-specific addressins.

174. The method of aspect 172, wherein said molecule capable of directing homing to the liver microenvironment are hepatic-specific integrins.

175. The method of aspect 105, wherein said hepatic primed mesenchymal stem cells are treated with hypoxia before administration in order to induce a state of hypoxic tolerance.

176. The method of aspect 105, wherein said hepatic primed mesenchymal stem cells are treated with acidic conditions before administration in order to induce a state of tolerance to inflammation associated acidity in the liver.

177. The method of aspect 105, wherein said hepatic primed mesenchymal stem cells are treated with 5-azacytidine to enhance therapeutic activity before administration.

178. The method of aspect 105, wherein said hepatic primed mesenchymal stem cells are treated with a ligand of the EPO receptor 1 to enhance therapeutic activity before administration.

179. The method of aspect 105, wherein said hepatic primed mesenchymal stem cells are treated with a c-met ligand to enhance therapeutic activity before administration.

180. The method of aspect 105, wherein said hepatic primed mesenchymal stem cells are administered intravenously.

181. The method of aspect 105, wherein said hepatic primed mesenchymal stem cells are administered intra-arterially.

182. The method of aspect 105, wherein said hepatic primed mesenchymal stem cells are administered intra-lymphatically.

183. The method of aspect 105, wherein said hepatic primed mesenchymal stem cells are administered intra-dermally.

184. The method of aspect 105, wherein said hepatic primed mesenchymal stem cells are administered into the portal vein.

185. The method of aspect 105, wherein said hepatic primed mesenchymal stem cells are administered into the portal artery.

186. The method of aspect 105, wherein prior to administration of said hepatic primed mesenchymal stem cells the canine liver is preconditioned to possess enhanced receptivity for said administered cells.

187. The method of aspect 186, wherein the liver is first treated with low infrared irradiation at a frequency and intensity to stimulate production of CXCL!2 ligand in the hepatic microenvironment by at least 10% compared to baseline.

188. The method of aspect 186, wherein the liver is first treated with low infrared irradiation at a frequency and intensity to stimulate production of CXCL!2 ligand in the hepatic microenvironment by at least 25% compared to baseline.

189. The method of aspect 186, wherein the liver is first treated with low infrared irradiation at a frequency and intensity to stimulate production of CXCL!2 ligand in the hepatic microenvironment by at least 100% compared to baseline.

190. The method of aspect 186, wherein the liver is first treated with extracorporeal pulse shock wave at a frequency and intensity to stimulate production of CXCL!2 ligand in the hepatic microenvironment by at least 10% compared to baseline.

191. The method of aspect 186, wherein the liver is first treated with extracorporeal pulse shock wave at a frequency and intensity to stimulate production of CXCL!2 ligand in the hepatic microenvironment by at least 25% compared to baseline.

192. The method of aspect 186, wherein the liver is first treated with extracorporeal pulse shock wave at a frequency and intensity to stimulate production of CXCL!2 ligand in the hepatic microenvironment by at least 100% compared to baseline.

193. The method of aspect 105, wherein exosomes are generated from canine inducible pluripotent stem cells and administered as a therapeutic for treatment of liver failure.

194. The method of aspect 105, wherein exosomes are generated from canine inducible pluripotent stem cell derived mesenchymal stem cells and administered as a therapeutic for treatment of liver failure.

195. The method of aspect 105, wherein exosomes are generated from canine inducible pluripotent stem cell derived mesenchymal stem cell that are differentiated into hepatocyte progenitors and administered as a therapeutic for treatment of liver failure.

196. The method of aspects 193 to 195, wherein said exosomes are between 50 nm to 300 nm in size.

197. The method of aspects 193 to 195, wherein said exosomes are between 100 nm to 300 nm in size.

198. The method of aspects 193 to 195, wherein said exosomes are between 150 nm to 250 nm in size.

199. The method of aspects 193 to 195, wherein said exosomes express CD63.

200. The method of aspects 193 to 195, wherein said exosomes express miR155.

201. The method of aspects 193 to 195, wherein said exosomes express CD8.

202. A therapeutic composition for stimulation of canine regeneration comprising exosomes derived from induced pluripotent stem cells.

203. The therapeutic composition of aspect 202, wherein said exosomes are concentrated by means of a solid substrate possessing selective affinity for said canine induced pluripotent stem cell exosomes.

204. The method of aspect 203, wherein said solid substrate contains one or more lectins.

### DETAILED DESCRIPTION OF THE INVENTION

In one embodiment, canine MSC are generated from induced pluripotent stem cells (iPSC). Similar to human iPSC, canine iPSCs are capable of generating embryoid bodies (EBs).

The term “stem cells” refers to master cells capable of being regenerated in a non-limiting manner to form specialized cells of tissues and organs. Stem cells are pluripotent or multipotent cells that can develop. Stem cells can divide into two daughter stem cells, or one daughter stem cell and one derived (‘transit’) cell, and then proliferate into cells in a mature and intact form of tissue. Such stem cells may be classified by various methods. One of the most commonly used methods is according to the differentiation ability of stem cells, and stem cells can be classified into pluripotent stem cells capable of differentiating into three germ layers, multipotent stem cells which are limited to differentiation into specific germ layers or higher, and unipotent stem cells capable of differentiating into specific germ layers only.

The term “pluripotent stem cells” refers to stem cells having totipotency capable of differentiating into all three germ layers constituting a living body, and generally, embryonic stem cells (ESCs) and induced pluripotent stem cells (iPSCs) correspond thereto. Adult stem cells may be classified into multipotent or unipotent stem cells.

The term “mesenchymal stem cells” refers to stem cells possessing multipotency capable of differentiating into cells such as adipocytes, osteocytes, chondrocytes, myocytes, nerve cells, and cardiomyocytes.

The term “differentiation” refers to a phenomenon in which a cell's structure or function is specialized during cell division, proliferation and growth. Pluripotent mesenchymal stem cells differentiate into progenitor cells of limited lineage (for example, mesodermal cells) and then may further differentiate into other forms of progenitor cells (for example, osteoblasts, and the like), and then may differentiate into terminal differentiated cells (for example, adipocytes, osteocytes, chondrocytes, and the like) that play a characteristic role in specific tissues (for example, bone, and the like).

The term “embryoid body (EB)” refers to an aggregate of pluripotent stem cells produced to induce the differentiation of pluripotent stem cells. In the present invention, the “mature embryoid body” is an aggregate of pluripotent stem cells, that is, an embryoid body at a state in which the embryoid body repeatedly divides through suspension culture and becomes larger in size, and the mature embryoid body in the present invention is used as a material for inducing differentiation into mesenchymal stem cells.

The term “cell therapeutic agent” refers to a drug used for the purpose of treatment, diagnosis, and prevention, by using a cell or tissue prepared through isolation from a human, culture and specific manipulation (US FDA regulations), and specifically, it refers to a drug used for the purpose of treatment, diagnosis, and prevention through a series of actions of in vitro multiplying and sorting living autologous, allogenic and xenogenic cells or changing the biological characteristics of cells by other methods for the purpose of recovering the functions of cells or tissues. Cell therapeutic agents are broadly classified into somatic cell therapeutic agents and stem cell therapeutic agents according to the degree of cell differentiation, and the present invention particularly relates to a stem cell therapeutic agent.

The term CD105 is a specific surface markers of mesenchymal stem cells, and according to a report by Duff S E et al. in 2003, it was reported that CD105 plays an important role in vascular regeneration by mesenchymal stem cells (The FASEB Journal 2003; 17(9):984-992). In addition, CD105 is reduced after not only differentiation of mesenchymal stem cells into osteocytes (Levi B et al., The Journal of Biological Chemistry. 2011; 286(45): 39497-39509), but also differentiation of mesenchymal stem cells into cells such as osteocytes, chondrocytes, and adipocytes, and thus is known to play an important role in maintaining the stemness of mesenchymal stem cells (Jin H J et al., BBRC 2009; 381(4):676-681).

The term “canine” refers to omnivorous animals, including dogs, wolves, foxes, coyotes, jackals, and Korean wolves, which are all digitigrade animals. The canine animals are broadly divided into Canini and Vulpini. Canini includes Chrysocyon brachyurus, Canis adustus, Canis mesomelas, Canis lupus familiaris, Canis lupus dingo, Canis rufus, Canis simensis, Canis pallipes, Canis latrans, Canis aureus, Cerdocyon, Speothos, Cuon alpinus, Lycaon pictus, Atelocynus microtis, Dusicyon australis, Lycalopex culpaeus, Lycalopex fulvipes, Lycalopex griseus, Lycalopex gymnocercus, Lycalopex sechurae, Lycalopex vetulus and the like. Vulpini includes Vulpes lagopus, Vulpes vulpes, Vulpes velox, Vulpes macrotis, Vulpes corsac, Vulpes chama, Vulpes pallid, Vulpes bengalensis, Vulpes ferrilata, Vulpes cana, Vulpes ruppelli, Vulpes zerda, Urocyon cinereoargenteus, Urocyon littoralis, Urocyon sp and the like, and there are other animals such as Otocyon megalotis, Nyctereutes procyonoides and the like.

Numerous conditions that affect dogs could be amenable to treatment with stem cell based therapies. Conventional canine stem cell therapies are currently limited to autologous sources. This presents the issue of needing tissue from animals that often are diseased, as well as the impossibility of treating genetic diseases. In the present invention, aims to solve the problems associated with autologous canine cell therapies by ensuring allogeneic cell safety and mass production in order to commercialize the mesenchymal stem cells induced to differentiate from canine pluripotent stem cells such as iPSC.

In one embodiment the invention provides the finding that a single spheroidal embryoid body can be allowed to be formed by intercellular aggregation of iPSC, the quality difference between mature embryoid bodies is minimized by suspension-culturing such embryoid bodies to form mature embryoid bodies uniform in shape and size, and the resulting efficiency and consistency of differentiation into mesenchymal stem cells were improved. The mesenchymal stem cells thus prepared showed an exceptional effect in which the mesenchymal stem cell characteristics can be stably maintained even after repeated long-term subcultures of 20 or more passages. That is, the present invention provides canine pluripotent stem cell-derived mesenchymal stem cells which are safe without contamination with a foreign cell and a foreign animal-derived material, have uniform quality through the formation of mature embryoid bodies uniform in shape and size using spheroidal embryoid bodies, and can be mass produced because the stability of subculture is excellent for a long period of time. Therefore, the mesenchymal stem cells with enhanced productivity and safety prepared by the method of the present invention make it possible to continuously supply a large amount of mesenchymal stem cells required in the fields of regenerative medicine and cell therapy.

In one embodiment of the invention, a methodology is disclosed for preparing canine mesenchymal stem cells from induced pluripotent stem cells comprising: Firstly culturing canine iPSC cells in a serum-free pluripotent stem cell culture medium without feeder cells to obtain a colony of canine iPSC cells and isolating said cells from the colony; Secondly, allowing for formation of single spheroidal embryoid bodies by suspending the isolated iPSC cells in an embryoid body formation medium, and then culturing the isolated iPSC cells such that the cells are aggregated; Thirdly, allowing the formation of mature embryoid bodies by suspension-culturing embryoid bodies in an embryoid body maturation medium; Fourthly, inducing differentiation into mesenchymal stem cells by adherently culturing the embryoid bodies in a xeno-free and serum-free mesenchymal stem cell culture medium; and Fifthly, allowing the proliferation and culturing of the differentiated canine mesenchymal stem cells in a xeno-free and serum-free mesenchymal stem cell culture medium while maintaining the identity of mesenchymal stem cells.

In general, a support is required to maintain an undifferentiated state in culturing canine pluripotent stem cells in the related art, and as a canine pluripotent stem cell support in the related art, murine embryo-derived fibroblasts have been preferentially used. However, since the influx of various pathogens between different species is recognized as a problem when trying to use pluripotent stem cells clinically, the potential as a support for various canine-derived cells has been reported as an alternative to the problem. However, it is impossible to overcome disadvantages in that it is also impossible to completely exclude heterologous pathogens, foreign factors (for example, bFGF, IGF, ACTIVIN, and the like) are essential for maintaining an undifferentiated state, and it is impossible to continuously supply the canine-derived cells for long-term culture, and the like.

However, in the method of the present invention, an undifferentiated state can be maintained even when a culture vessel coated with vitronectin is used without xeno feeder cells, and canine pluripotent stem cells are cultured in a serum-free medium.

In the present invention, the pluripotent stem cells in Step (a) are preferably cells cultured in a culture vessel coated with a canine-derived extracellular matrix, or a synthetic material capable of replacing the extracellular matrix, but are not limited thereto. The canine-derived extracellular matrix is preferably vitronectin, collagen, or laminin, and the extracellular matrix that does not contain an animal-derived component other than canine is preferably an animal component-free Matrigel, and the synthetic material capable of replacing the extracellular matrix is preferably heparan sulfate proteoglycan, but they are not limited thereto. That is, the method of the present invention may be characterized in that it is performed in a medium free of xeno feeder cells, cytokines and xenogeneic materials in all steps. That is, the method of the present invention may be characterized in that it is performed in a feeder cell-free, xeno-free and serum-free environment.

In addition, in the related art, DMEM/F12 containing KSR, NEAA, β-mercaptoethanol, and bFGF may be used as a pluripotent stem cell culture medium, but in the present invention, a serum-free pluripotent stem cell culture medium is used. In the present invention, the serum-free medium for culturing the canine iPSC in Step (a) may be TeSR-Essential 8 (TeSR-E8) medium, TeSR-2 medium or StemMACS iPS-Brew XF, but is not limited thereto. (b) forming single spheroidal embryoid bodies by suspending isolated pluripotent stem cells in embryoid body formation medium, and then culturing isolated pluripotent stem cells such that pluripotent stem cells are aggregated;

In the present invention, it is characterized in that mature embryoid bodies uniform in shape and size are used to induce differentiation from canine iPSC cells to mesenchymal stem cells. In the method in the related art, it was difficult to expect consistent differentiation efficiency into mesenchymal stem cells because it was not possible to produce embryoid bodies uniform in shape and size, but in the present invention, these technical limitations were overcome by allowing a single spheroidal form of canine iPSC cells to be formed by intercellular aggregation and suspension-culturing these embryoid bodies to form mature embryoid bodies uniform in shape and size. Through this, exceptionally excellent mesenchymal stem cells, which improved the differentiation efficiency of canine derived mesenchymal stem cells and maintained stem cell characteristics for a long time of time even after repeated subcultures, were prepared. In the method in the related art (WO 2011052818), pluripotent stem cells were suspension-cultured during embryoid body formation, but in the present invention, in order that the pluripotent stem cells aggregate with each other to form a single spheroidal embryoid body, pluripotent stem cells were inoculated onto a lid of a culture vessel and then the culture vessel was inverted upside down to perform hanging drop culture for 24 hours such that the cells could aggregate by gravity, and the thus-formed cell aggregates, that is, embryoid bodies were suspension-cultured and matured into mature embryoid bodies. Embryoid body prepared by the method of the present invention have a uniform size of 300 to 500 and the shape is also constant as a spheroid. In addition, it can be seen that the mesenchymal stem cells differentiation-induced from the mature embryoid body thus prepared have a constant differentiation efficiency and a constant size of the mesenchymal stem cells. However, the mesenchymal stem cells differentiation-induced from the embryoid body prepared by the method in the related art are not uniform in differentiation efficiency as well as in cell shape.

Moreover, the mesenchymal stem cells differentiation-induced from mature embryoid bodies uniform in shape and size surprisingly showed 90% or more expression of the mesenchymal stem cell surface markers CD29, CD44, CD73, and CD105 by passage 20. In addition, the expression of cell surface markers for a hematopoietic stem cell-specific surface marker CD45, an MHC class type II marker HLA-DR, and pluripotent stem cell-specific surface markers SSEA-3, TRA-1-60, and TRA-1-81 was found to be 2% or less. That is, since the mesenchymal stem cells prepared by the method of the present invention can be subcultured for a long period of time and maintain high purity, it is possible to mass-produce stem cells that can be used as a cell therapeutic agent. When the mesenchymal stem cells prepared by the method in the related art meet the criteria for use as a cell therapeutic agent, the cells are aged and modified and thus are no longer suitable for achieving the purpose as a cell therapeutic agent. In the end, there was a disadvantage in that cells applied to clinical trials and cells undergoing quality evaluation had to be different. However, since the mesenchymal stem cells of the present invention are not modified even after repeated subcultures for a long period of time for use as a cell therapeutic agent and maintain the quality characteristics of the mesenchymal stem cells, the mesenchymal stem cells of the present invention are very useful for commercializing a cell therapeutic agent.

Although methods for controlling the size of an embryoid body or methods for forming embryoid bodies uniform in size through a hanging drop means, and the like are known, it is known that the formation of embryoid bodies uniform in size affects cell differentiation efficiency, but the effect on the inhibition of senescence or cell modification of mesenchymal stem cells differentiation-induced from embryoid bodies uniform in size is not known at all. However, in the present invention, it was confirmed that mature embryoid bodies uniform in shape and size are formed to maintain the mesenchymal stem cell characteristics of the mesenchymal stem cells differentiation-induced therefrom for a long period of time until passage 20. In particular, the invention discloses that the mesenchymal stem cells differentiation-induced from embryoid bodies prepared by the method in the related art. That is, the stem cells prepared by the method in the related art do not maintain the characteristics of mesenchymal stem cells until passage 20, but the stem cells of the present invention can maintain the characteristics of mesenchymal stem cells for a long period of time until passage 20 through the formation of embryoid bodies uniform in size.

Therefore, the present invention describes the preparation of canine mesenchymal stem cells which are highly safe by employing a feeder cell-free, xeno-free, and serum-free culture environment to solve the problem of contamination with a foreign animal-derived material, and simultaneously prepared canine iPSC cell-derived mesenchymal stem cells having exceptional effects of improving the differentiation efficiency to mesenchymal stem cells by utilizing spheroidal embryoid bodies to form mature embryoid bodies uniform in shape and size and stably maintaining mesenchymal stem cell characteristics even after long-term subcultures, such as 20 or more passages. As used herein, the “spheroidal embryoid body” refers to a round spherical cell aggregate, wherein the spheroidal form is often expressed as a spheroid form.

In the present invention, any culture method capable of inducing intercellular aggregation of pluripotent stem cells can be used for the embryoid body in Step (b). Any culture method capable of preparing an aggregate of pluripotent stem cells, that is, a single spheroidal embryoid body is possible and not limited. For example, the embryoid body in Step (b) may be formed by hanging drop culture, or culture using a V-shape tube, a round shape 96 well plate, or a conical tube. In the present invention, any medium capable of inducing cell aggregation between pluripotent stem cells can be used as the embryoid body formation medium in Step (b). For example, the embryoid body formation medium in Step (b) may be an Aggrewell EB formation medium, Gibco Essential 6 Medium, CTS Essential 6 Medium, or TeSR-E6. The culture in Step (b) may be performed for 18 hours to 30 hours, for example, 20 hours to 28 hours, 22 hours to 26 hours, such as 24 hours. (c) forming mature embryoid bodies by suspension-culturing embryoid bodies in embryoid body maturation medium; Step (c) is a step in which the embryoid body is grown while being suspension-cultured, and the mature embryoid body obtained in this step is characterized by having a uniform shape and size.

The fact that mature canine embryoid bodies have “uniform shape and size” means that mature embryoid bodies obtained by suspension-culturing spheroidal embryoid bodies are uniform in shape and size. The shape of the mature embryoid bodies is spheroidal like the embryoid body, and the size of the mature embryoid body is uniform within ±15% of the average size of all mature embryoid bodies. That is, assuming that the average size of the mature embryoid bodies is 100%, the minimum size of the mature embryoid body is within 90% and the maximum size of the mature embryoid body is within 120%, and the size thereof is quite uniform. Preferably, the size of the mature embryoid body is uniform with a size of ±10% of the average size of all mature embryoid bodies. In this case, assuming that the average size of the mature embryoid bodies is 100%, the minimum size of the mature embryoid body is 90%, and the maximum size of the mature embryoid body is within 110%. Although the average size of the mature embryoid bodies may vary depending on the maturation culture conditions and period of the embryoid body, the average size of the mature embryoid bodies suitable for induction of differentiation into stem cells may be 350 to 450 μm, for example, 380 to 420 μm. Preferably, the mature embryoid body has a uniform size of 300 to 500 μm. In the present invention, the suspension culture in Step (c) is performed using an embryoid body maturation medium for embryoid body maturation. A generally known embryoid body maturation medium may be used, and is not particularly limited. Although not limited thereto, in an exemplary embodiment of the present invention, the embryoid body maturation medium in Step (c) may be a basic medium supplemented with knock out serum replacement (KSR), non-essential amino acids (NEAA) and β-mercaptoethanol. The basic medium may be DMEM/F12, alpha MEM, Ham's F12 media, or DMEM, but is not limited thereto. The suspension culture in Step (c) may be performed for 10 to 18 days, for example, 12 to 16 days, such as, 14 days, but is not limited thereto. The next step is inducing differentiation into mesenchymal stem cells by adherently culturing embryoid bodies in xeno-free and serum-free mesenchymal stem cell culture medium. In the case of inducing differentiation from canine pluripotent stem cells to mesenchymal stem cells, the induction of differentiation is generally initiated by adding a cytokine, for example, bone morphogenetic protein (BMP), and the like, from the outside, but in the present invention, it was confirmed that differentiation into mesenchymal stem cells was naturally induced without the addition of BMP and the like. In the method in the related art (WO 2011052818), a DMEM medium containing FBS was used for differentiation of mesenchymal stem cells, and an EGM2-MV medium containing FBS was also used for proliferation culture. However, in the present invention, proliferation and culture of mesenchymal stem cells were performed in a xeno-free and serum-free environment. In the present invention, the canine mesenchymal stem cell culture medium used in Step (d) may be a xeno-free and serum-free medium containing L-glutamine. The total concentration of L-glutamine in the medium is preferably used in accordance with 2 to 4 mM, but is not limited thereto. Examples of the xeno-free and serum-free mesenchymal stem cell culture medium in Step (d) include a Stempro SFM xeno-free medium, a PRIME-XV MSC expansion XSFM medium, a Mesenchymal-XF Expansion medium, an MSC Nutristem XF medium, a StemMACS MSC expansion media kit XF, or a medium containing 5 to 20% canine platelet lysate instead of FBS, but are not limited thereto.

Further, in the present invention, the induction of differentiation into canine mesenchymal stem cells in Step (d) may be 12 to 20 days, for example, 14 to 18 days, such as 16 days, but is not limited thereto. In an exemplary embodiment of the present invention, differentiation of mesenchymal stem cells was induced in a Stempro MSC SFM Xeno-free medium that does not contain differentiation inducing factors and serum. (e) proliferating and culturing the differentiated mesenchymal stem cells in xeno-free and serum-free mesenchymal stem cell culture medium while maintaining identity of mesenchymal stem cells. Step (e) is a step of proliferating and culturing differentiation-induced mesenchymal stem cells. In order to commercialize a cell therapeutic agent containing canine mesenchymal stem cells, it is a very important issue whether the mesenchymal stem cells obtained in this step are secured in a sufficient amount, while simultaneously maintaining the identity of the canine mesenchymal stem cells, that is, the characteristics as mesenchymal stem cells. In the present invention, in the proliferation culture of the differentiation-induced mesenchymal stem cells, the mesenchymal stem cells are cultured in a xeno-free medium to which additional differentiation inducing factors and fetal bovine serum (FBS) are not added. However, in some embodiments, canine monocyte conditioned media may be utilized. In other embodiments, canine platelet lysate may be utilized as an expansion media. As in Step (d), in the present invention, the mesenchymal stem cell culture medium used in Step (e) may be a xeno-free and serum-free medium containing L-glutamine. The total concentration of L-glutamine in the medium is preferably used in accordance with 2 to 4 mM, but is not limited thereto.

Examples of the xeno-free and serum-free mesenchymal stem cell culture medium in Step (e) include a Stempro SFM xeno-free medium, a PRIME-XV MSC expansion XSFM medium, Mesenchymal-XF Expansion medium, an MSC Nutristem XF medium, a StemMACS MSC expansion media kit XF, or a medium containing 5 to 20% canine platelet lysate instead of FBS, but are not limited thereto.

In an exemplary embodiment of the present invention, a Stempro MSC SFM medium to which FBS was not added was used as a mesenchymal stem cell proliferation medium, but the present invention is not limited thereto, and a medium that does not contain a heterologous material such as a heterologous protein (xeno-free) may be used. As described above, in order to use mesenchymal stem cells as a cell therapeutic agent, it is necessary to first supply a sufficient amount of cells, and for this purpose, the subculture of canine mesenchymal stem cells is required. However, when the subculture is continued, there is a problem in that the mesenchymal stem cells age and lose their division ability and lose their activity (differentiation ability). In this regard, in the present invention, it was confirmed that the characteristics and activity of the mesenchymal stem cells could be maintained for 20 or more passages during ex vivo culture even in a xeno-free and serum-free medium. That is, the mesenchymal stem cells prepared by the method of the present invention can maintain the characteristics of the mesenchymal stem cells for a long period of time, and thus can be used as a cell therapeutic agent through mass production. These characteristics may be achieved through a xeno-free and serum-free mesenchymal stem cell preparation environment and the preparation of embryoid bodies uniform in size.

Classically, both human and canine mesenchymal stem cells are defined by a uniform spindle-shaped fingerprint pattern and the expression levels of basic cell surface markers such as CD73(+), CD105(+), CD34(−), and CD45(−), and can be differentiated into osteocytes, chondrocytes, adipocytes, and the like. In the present invention, the mesenchymal stem cells in Step (e) may be characterized by being mesenchymal stem cells possessing multipotency capable of differentiating into cells selected from the group consisting of adipocytes, osteocytes, chondrocytes, myocytes, nerve cells and cardiomyocytes. In an exemplary embodiment of the present invention, in order to confirm the stem cell characteristic persistence (consistency) according to the subculture of pluripotent stem cell-derived mesenchymal stem cells, a change in cell surface markers up to passage 20 was comparatively analyzed. As a result of comparatively analyzing the expression of cell surface marker for mesenchymal stem cell surface markers CD29, CD44, CD73, and CD105, a hematopoietic stem cell-specific surface marker CD45, an MEW class type II marker HLA-DR, and pluripotent stem cell-specific surface markers SSEA-3, TRA-1-60 and TRA-1-81 from passage 12 to passage 20, it was confirmed that the expression of mesenchymal stem cell surface markers CD29, CD44, CD73, and CD105 was maintained at 90% or more until passage 20 (Table 1). Furthermore, in the pluripotent stem cell-derived mesenchymal stem cells prepared by the method in the related art (WO 2011052818), the expression of CD105 was shown to be less than 50% in passage 6 cells, and in particular, it was confirmed that the expression was reduced to 26.6% after passage 12. That is, the stem cells prepared by the method in the related art do not maintain the characteristics of mesenchymal stem cells until passage 20, but the stem cells of the present invention can maintain the characteristics of mesenchymal stem cells for a long time up to passage 20 through the formation of a mature embryoid body having a uniform shape and size.

In the present invention, the mesenchymal stem cells in Step (e) may be characterized by being mesenchymal stem cells expressing CD29(+), CD44(+), CD73(+) and CD105(+) cell surface markers.

In the present invention, the expression of the cell surface marker is preferably maintained at 90% or more in mesenchymal stem cells of 20 or more passages, more preferably, the expression of a CD105(+) cell surface marker in mesenchymal stem cells of 20 or more passages is maintained at 90% or more, but is not limited thereto. In the present invention, the canine mesenchymal stem cells in Step (e) may be characterized by being mesenchymal stem cells of CD34(−), CD45(−), HLA-DR(−), TRA-1-60(−), and TRA-1-81(−). In another aspect, the present invention relates to mesenchymal stem cells differentiated from canine iPSC cells prepared by the method.

In one embodiment, canine iPSC are removed from feeder cell/suspension culture and transferred into non-adherent culture vessels to permit formation of EBs. In one embodiment, iPSC are dissociated from adherent culture by treatment with Accutase. In some embodiments, Accutase is diluted with phosphate buffered saline (PBS). Cells cultured as EBs are treated with differentiation factors, specific differentiation factors include VEGF and BMP4.

The state of clinically applied canine regenerative medicine is significantly behind human regenerative medicine, in part, due to lack of practical universal donor cell populations. In contrast to other species, freshly explanted canine MSC appear to be more difficult to expand in vitro. This may be because of a more “aged” population of these cells in the canine species. Accordingly, the invention provides a simplified means for selectively promoting the formation of a substantially homogenous population of mesenchymal stem cells derived from canine induced pluripotent stem cells. The advantage of using such an immature cellular population is that it offers the opportunity to create more potent MSC which still maintain ability to act across allogeneic barriers. The invention teaches that the culturing of induced pluripotent stem cells under conditions conducive for the formation of embryoid bodies. The embryoid bodies are propagated in mesenchyme-specific medium and digested to form mesodermal cells. The mesodermal cells are further cultured in mesenchyme-specific medium to form a substantially homogenous population of mesenchymal stem cells. In some embodiments generated mesoderm cells are harvested using isolation techniques in order to selectively obtain more immature cells.

One method of obtaining more immature cells is in vitro treatment with cytotoxic agents. Early stem cells possess drug efflux pumps, which allow survival of immature MSC cells while ablating more mature cells. In an related embodiment, the substantially homogenous population of mesenchymal stem cells can be further propagated in the presence of an effective amount of 1.25(OH)2D3 to produce a subpopulation of bone-precursor cells including pre-osteoblasts and osteoblasts. These cells are competent to mineralize and thus form bone. In a related embodiment, the method can be further simplified by directly treating embryoid bodies derived from iPSC cells, or mesodermal cells with an effective amount of 1.25(OH)2D3 to facilitate formation of mesenchymal stem cells. Direct treatment of embryoid bodies with 1.25(OH)2D3 permits mesenchymal stem cell cultures to be created that are relatively homogenous, having in excess of 90% of the cells being mesenchymal stem cells, in a relatively rapid and convenient protocol. Normally it takes about 10 to 12 days to culture canine iPSC cells into embryoid bodies. Then, to go from embryoid bodies to a homogenous canine MSC culture using this new method takes only a total time period of less than 10 days, and typically 6 to 8 days. When the efficiency of this process is combined with the process to induce the MSCs to become bone-forming osteoblasts described below (which takes 7 to 14 days), it is possible to go from canine ES cells to bone-forming osteoblasts in less than 34 days, and in as few as 23 days, of culture to achieve both differentiation and mineralization.

In contrast to human cells of the MSC lineage, canine MSC appear to possess increased levels of c-kit and decreased levels of CD73. This is an important distinguishing feature. Furthermore, the invention teaches that administration of hepatocyte growth factor during the iPSC mediated creation of EBs results in increase canine MSC numbers, which is not observed when this procedure is performed on human cells.

In a specific embodiment, the invention provides a method for producing a substantially homogenous population of hepatocytes, cardiomyocytes, pre-osteoblast and osteoblast cells useful for canine treatments. Dog iPSC cells are cultured into embryoid bodies which are then cultured in the presence of a mesenchyme-specific medium that favors the optimal propagation of mesenchymal stem cells as described above to form a homogenous population of MSCs. The MSC population is further cultured in osteogenic medium, where effective amounts of hepatic, cardiomyocyte or osteoblast differentiation agents are introduced into the medium in a sequence-specific manner. This is performed by initially introducing 1.25(OH)2D3, dexamethasone, retinoic acid, or combinations thereof, to the osteogenic culture medium followed by the addition of ascorbic acid and β-glycerophosphate. This promotes differentiation of the MSCs into a substantially homogenous population of cells expressing gene markers and exhibiting cell morphology characteristic of bone-forming cells. Accordingly, the remarkable uniformity and functional properties of the cells produced by the methods of the invention make them valuable (1) for preparing and characterizing canine MSC involved in the turnover and repair of bone; (2) for developing new therapeutic models; and (3) as a tool for studying mesenchymal tissues in vitro.

In another embodiment, the invention provides a substantially homogenous population of mesenchymal stem cells derived from canine iPSC cells. The homogenous population is composed of no less than 60% and preferably 90% mesenchymal stem cells exhibiting spindle shaped morphology. The MSC population may be further cultured in the presence of an effective amount of 1.25(OH)2D3 to selectively promote the formation of bone precursor cells, including pre-osteoblasts and osteoblasts. The pre-osteoblasts and osteoblasts secrete matrix proteins and undergo mineralization. Also, the pre-osteoblasts or osteoblasts are characterized by the expression of early transcription factors, such as, but not limited to Cbfa-1, Msx2, and D1x5. These cells are characterized by the expression of osteoblast specific genes, such as, but not limited to osteopontin, osteonectin, and osteocalcin.

Canine MSCs can be induced to proliferate and differentiate in different ways and to treat different conditions. In some embodiments hepatic failure is treated by intrahepatic administration. In other embodiments, conditioned media of canine MSCs is used as a therapeutic agent. Said conditioned media may be harvested from MSCs in standard tissue culture, or may be collected after stimulation of the MSCs with a “priming” signal. Said priming signal may be a “danger” signal such as a toll like receptor agonist, a STING agonist, or may be temporary, sublethal hypoxia.

The invention teaches that canine MSCs can be cultured on a substrate coated with an appropriate material conducive to growth of the desired cell phenotype, or MSCs can be cultured in a medium containing a variety of components to induce growth. For example, in accordance with the invention, MSCs may be cultured in mesenchyme-specific medium. The term “mesenchyme-specific medium” as used herein refers to a culture medium containing MesenCulti™ Basal Medium and 10% FBS for human mesenchymal stem cells. This medium which is made of McCoy's base, specifically McCoy's 5A medium (modified) supplemented with 2.0 mM L-Glutamine is essentially used for the expansion and differentiation of human mesenchymal stem cells. Although all of the components of the MesenCulti™ Basal Medium are not disclosed by the manufacturer, applicants believe that the following is a non-limiting list of suitable components: non-essential amino acids, ribonucleosides, deoxyribonucleosides, L-glutamine, sodium bicarbonate, and Vitamin B12. Accordingly, it is envisioned that other media, besides McCoy's base, containing the above-mentioned components can be utilized to facilitate MSC differentiation. To verify that other mesenchyme-specific media will work as well, we have also tested alpha MEM medium (Earl's) with glutamine and nucleosides added, and it worked as well as the MesenCult medium. Another useful mesenchyme-specific medium is DMEM with glucose and glutamine, although it may lead to a slightly decreased yield of embryoid bodies. Characterized or defined serum products from various manufacturers can be used in place of the pre-tested serum in the media below as well.

Furthermore, to optimize the expansion of canine mesenchymal cells, Fetal Bovine Serum (FBS) pre-tested for optimal growth of hMSCs (available through Stem Cell Technologies, Vancouver, Canada) may be added to the MesenCulti™ Basal Medium. Although the components of FBS are numerous due to the natural source of this material, it is believed that the following is a non-limiting list of suitable components that may influence the growth of MSCs: transferrins, cytokines/growth factors such as insulin growth factors (IGFs), basic fibroblast growth factor (bFGF), stem cell factor (SCF), bone morphogenic proteins (BMPs) including and not limited to BMP 2, 4, 7, 9 and 12. These components have been pretested and selected for their ability to optimally initiate and maintain human mesenchymal cell proliferation. In some embodiments, tissue culture media utilized includes an ES medium [40% Dulbecco's modified Eagle medium (EMEM), 40% F12 medium (Sigma), 2 mM L-glutamine or GlutaMAX (Sigma), 1% non-essential amino acid (Sigma), 0.1 mM 3-mercaptoethanol (Sigma), 15-20% Knockout Serum Replacement (Invitrogen), 10 μg/ml of gentamicin (Invitrogen), and 4-10 ng/ml of bFGF (FGF2) factor](hereinafter referred to as ES medium), a conditioned medium that is the supernatant of a 24-hr culture of mouse embryonic fibroblasts (hereinafter referred to as MEF) on an ES medium lacking 0.1 mM 3-mercaptoethanol and which is supplemented with 0.1 mM 3-mercaptoethanol and 10 ng/ml of bFGF (FGF2) (this medium is hereinafter referred to as MEF conditioned ES medium), an optimum medium for iPS cells (iPSellon), an optimum medium for feeder cells (iPSellon), StemPro (registered trademark) hESC SFM (Invitrogen), mTeSR1 (STEMCELL Technologies/VERITAS), an animal protein free, serum-free medium, named TeSR2 [ST-05860](STEMCELL Technologies/VERITAS), ES/iPS cells (ReproCELL), ReproStem (ReproCELL), ReproFF (ReproCELL), and ReproFF2 (ReproCELL). It is envisioned that Matrigel™, laminin, collagen (especially collagen type I), glycosaminoglycans, osteocalcin, and osteonectin may all be suitable as an extracellular matrix, by themselves or in various combinations. Also potentially suitable for growing osteoblast lineage cells are gel-derived glasses, silica gels, and sol-gel-derived titania (Saravanapavan et al., (2001) J. Biomed Mater. Res. 54:608; Dieudonne et al., (2002) Biomaterials 34:3041.

Furthermore, the canine MSCs produced by the methods of this invention may be characterized according to a number of phenotypic criteria. For example, relatively undifferentiated mesenchymal cells can be recognized by their characteristic mononuclear ovoid, stellate shape or spindle shape, with a round to oval nucleus. The oval elongate nuclei typically have prominent nucleoli and a mix of hetero- and euchromatin. These cells have little cytoplasm but many thin processes that appear to extend from the nucleus. It is believed that canine MSCs will typically stain for one, two, three or more of the following markers: CD106 (VCAM), CD166 (ALCAM), CD29, CD44, GATA-4, and alkaline phosphatase, while being negative for hematopoietic lineage cell markers (CD14 or CD45). MSCs may also express STRO-1 as a marker.

The properties of canine MSCs that allow for therapeutic use include production of growth or angiogenic factors, or also include their ability to form other tissues. The ability of canine MSC to “differentiate” or “differentiated” refers to a cell that has progressed further down a developmental pathway or lineage than the cell it is being compared with.

In the method of the present invention for differentiating an induced hepatic stem cell into induced hepatic progenitor cells or hepatocytes, the induced hepatic stem cell, before it is cultured in the presence of a TGF-β inhibitor, may be subjected to preliminary culture in a pluripotent stem cell culture medium in the presence of a feeder cell and only then the induced hepatic stem cell is cultured in the presence of a TGF-β inhibitor. As a result of this preliminary culture, the induced hepatic stem cell is brought into a preparatory stage for differentiation into induced hepatic progenitor cells or hepatocytes.

The culture described above induces differentiation of the induced hepatic stem cell into induced hepatic progenitor cells, and by further continuing the culture, differentiation of the induced hepatic progenitor cells into hepatocytes is induced.

Canine liver failure is a substantial problem. In some embodiments, the invention teaches that iPSC derived hepatic stem cell can be utilized for treatment of hepatic dysfunction or failure. Said iPSC derived hepatic stem cells are characterized in that they express at least the POU5F1 (OCT3/4) gene, the NANOG gene, and the SOX2 gene. By culturing this cell in the presence of a TGF-β inhibitor in accordance with the method of the present invention, differentiation into induced hepatic progenitor cells is first induced. The induced hepatic progenitor cell is characterized in that the expression of the hepatic stem/progenitor cell marker DLK1 or AFP gene as a gene associated with the properties of hepatocytes is increased markedly and that the expression of the hepatocyte markers ALB gene, AAT gene, TTR gene, FGG gene, AHSG gene, FABP1gene, RBP4 gene, TF gene, or APOA4 gene is also increased markedly. The hepatic progenitor cell is also characterized by expression of genes selected from a group comprising of: a) POU5F1; b) NANOG; c) SOX2; or d) PIM1. In the present invention, the pluripotent stem cell derived canine liver progenitor cells obtained by the above-described method are further cultured continuously to induce differentiation into hepatocytes. The thus obtained hepatocytes are characterized in that the hepatic genes which were expressed in the induced hepatic stem cell in amounts substantially comparable (%8-8 times) to the levels expressed in the induced pluripotent stem cells are expressed in the hepatocyte in amounts even much smaller than the levels expressed in the induced hepatic stem cell, or their expression is substantially absent, and the hepatocytes are also characterized in that among the genes associated with liver differentiation.

In one embodiment, the hepatic stem/progenitor cell marker DLK1 or AFP gene is markedly decreased or substantially absent whereas the expression of the hepatocyte markers ALB gene, AAT gene, TTR gene, FGG gene, AHSG gene, FABP1gene, RBP4 gene, TF gene, and APOA4 gene is increased even more markedly in differentiated hepatic cells. It is also within the scope of the present invention that as the differentiation of the induced hepatic stem cell into induced hepatic progenitor cells is induced, at least one gene selected from among the SOX17 gene, the FOXA2 gene and the GATA4 gene which are characteristic of endodermal cells may become expressed, and as the differentiation of the induced hepatic progenitor cells into hepatocytes is induced.

In one embodiment of the invention, pluripotent stem cells of canine origin are used to generate mature functional hepatocytes directly, or through the intermediate step of going through defined endodermal population. The canine iPSC cell-derived hepatocyte precursors and mature hepatocytes may be used for direct cell therapy or utilized in an extracorporeal bioartificial liver device to treat liver failure. Additionally, iPSC cell-derived mature hepatocytes may replace isolated primary hepatocytes or hepatocarcinoma cell lines which are routinely used by the pharmaceutical industry for drug screening assays. Another area of application for the differentiated hepatocytes is in the production of therapeutically useful proteins.

The normally non-proliferative hepatocytes in the liver have a remarkable ability to regulate its growth mass. After a 30-60% partial hepatectomy, the rat liver can regenerate its original volume within two to three weeks (Michalopoulos et al., 276(5309) Liver Regeneration. Sci. 60-66 (1997)), while the human liver takes sixty days to regenerate (Wang et al., 100(S1) P.N.A.S. 11881-88 (2003)). Unfortunately this regenerative capacity is compromised with liver disease. Attempts to proliferate adult hepatocytes in vitro for reconstitution back into the diseased liver have not been successful. Although resident liver stem cells (also termed “oval cells”, “hepatocyte precursors”, or “hepatoblasts”) are present in rodents and show some capacity to proliferate and differentiated into mature hepatocytes (Wang et al., 2003; Rogler, 150(2) Am. J. Pathol. 591-602 (1997)), their existence in dogs is not yet known. A method other investigators have suggested for proliferating hepatocytes is through cell immortalization by transfecting adult hepatocytes with the large T antigen of the SV40 virus (Pfeifer et al., 90(11) P.N.A.S. 5123-27 (1993).

Development of protocols to induce endoderm from iPSC cells has severely lagged when compared with protocols for the ectodermal or mesodermal counterparts. As a result there is no adequate cell source for endodermal tissues derived from the iPSC stem cells. Advanced efforts to characterize endoderm in depth have suffered from several obstacles, including the lack of an endodermal cell line, scarcity of isolated endoderm from embryonic tissue, lack of control of differentiation, as well as the absence of any known cell surface markers for endoderm. Of the few available protocols available (Chang & Zandstra, 88(3) Biotechnol Bioeng, 287-98 (2004); Kubo et al., 131(7) Devel. 1651-62 (2004)), there are no reports of inducing a greater than 50% endoderm-enriched population, there are no reports of augmenting endodermal proliferation, nor are there any reports of controlling the endodermal population along one particular lineage.

In one embodiment of the invention, canine iPSC are used for differentiation along the hepatic lineage is to expose them to cues from the liver. For example, recent studies have shown that stem cells from mesenchymal and hematopoietic origin can be induced to become hepatocyte-like cells following coculture with liver cells (Jang et al., 6 Nat. Cell Biol. 532-39 (2004); Lange et al., 11 World J. Gastroenterol. 4497-504 (2005)). One group has shown that immature ES cells can be driven to differentiate into a hepatic phenotype following transplantation in partially hepatectomized nude mice (Imamura et al., 10 Tissue Eng. 1716-24 (2004)). Another group has shown that ES cell engraftment into the liver can correct factor IX deficiency in mice. (Fair et al., 102 P.N.A.S. U.S.A. 102, 2958-963 (2005)). Taken together, these studies suggest that liver-specific cues may direct differentiation toward a hepatic phenotype both in vivo and in vitro. The present invention provides for a rapid differentiation method to obtain a homogeneous endoderm-like cell population with 95% purity. The direct differentiation was achieved by culturing canine iPSC on top of a collagen sandwich of primary dog hepatocytes. The presence of adult hepatocytes, but not liver endothelial cells or fibroblasts, promotes the differentiation and the proliferation of iPSC cells into a strikingly uniform population of endoderm-like cells, expressing the major endodermal markers Forkhead box protein A2 (Foxa2, formerly HNF3β), SRY-box containing gene 17 (Sox17), and alpha-fetoprotein (AFP) (Zaret 209 Dev. Biol. 1-10 (1999); Zaret, 3 Nat. Rev. Genet. 499-512 (2002)). When these iPSC cell derived endoderm-like cells were re-plated on a feeder layer of 3T3-J2 fibroblasts, further proliferation and differentiation along the hepatocyte lineage was observed, demonstrating hepatic morphology, functionality, and gene and protein expression. Furthermore, by seeding a bioartificial liver (BAL) device with these iPSC-derived hepatocyte-like cells we demonstrate an increased survival of rats following D-galactosamine (GalN)-induced fulminant hepatic failure (FHF).

The invention teaches that the culture of canine iPSC cell on top of collagen-sandwiched primary canine hepatocytes stimulates their differentiation and proliferation into a homogeneous population of endoderm-like cells. In another embodiment canine hepatic cell lines are used as a substitute for primary cells. The endoderm-like cell population, which emerged within the first week of culture, had cubical cell morphology and bright cell borders and maintained a high level of gene and protein expression for the endoderm specific genes Foxa2, Sox17, and AFP (Zaret, 209 Dev. Biol. 1-10 (1999); Zaret, 3 Nat. Rev. Genet. 499-512, (2002)). This cell population is also negative for major mesoderm and ectoderm markers suggesting an induced endodermspecific differentiation. The invention teaches that this endoderm-like cell population could be further differentiated to hepatocyte-like cells, by subculturing the cells on a feeder layer of 3T3-J2 fibroblasts. The hepatocyte-like cell population that emerged had a hepatocyte morphology, expressed hepatocyte markers on the gene level, stained positive for albumin and CK-18 filaments, and secreted urea. During development, the first sign of liver morphogenesis is a thickening of the ventral endoderm, which occurs around embryonic day eight in the mouse (Zaret, 11 Curr. Opin. Genet. Devel. 568-74 (2001)). Little is known about the signals involved in initial endoderm formation and subsequent endoderm specification, but recent studies suggest a role for FGF, BMP, and activin. FGF, both acidic and basic, produced by the cardiac mesoderm was shown to induce the foregut endoderm to the hepatic lineage (Zaret, 2001), while BMP produced by the transversum mesenchyme was shown to increase levels of GATA4 (Wells & Melton, 15 Annu. Rev. Cell Devel. Biol. 393-410 (1999)). Activin was also shown to participate in the early induction of endoderm through smad2 signaling (Ball & Risbridger, 238 Devel. Biol. 1-12 (2001)). By embryonic day 8.5 in the mouse, definitive endoderm has formed the gut tube and expresses Foxa2. As liver morphogenesis progresses, Foxa2 positive cells proliferate to form the hepatic diverticulum while expressing AFP. Final maturation of these hepatic progenitors occurs when hepatic cords associate with the mesenchymal cells of the septum transversum, forming the liver sinusoids while expressing albumin and urea (Zaret, 2001).

A similar path of differentiation occurs during culture of embryoid bodies (EB), which is the most common method of ES cell differentiation (Hamazaki et al., 497 FEBS Lett. 15-19 (2001)). Normally the early cell populations that arise during initial ES cell differentiation consist of neuroectoderm, mesoderm, definitive endoderm, and extraembryonic endoderm. The bipotent mesendoderm may also be present (Kubo et al., 131 Devel. 131, 1651-1662 (2004); Tada et al., 132 Devel. 4363-74 (2005)). Although this mixed cell population does give rise to hepatocyte-like cells, usually in close proximity to cardiac-like tissue, the low yield makes it essential to purify the cell population to further explore their potential. In an effort to create a homogeneous cell population, several groups have studied endoderm differentiation in monolayer culture thereby exposing the ES cells to uniform cues from their microenvironment.

Several groups have shown the importance of collagen in the differentiation of pluripotent cells toward the hepatic phenotype. The differentiation of iPSC cells on three-dimensional collagen gels has not been previously investigated, however. The ability of three-dimensional collagen gels to induce and maintain epithelial morphology and function is well established, suggesting a similar enhancement of epithelial differentiation might occur during iPSC culture. Therefore, to create an environment conducive for hepatic differentiation the invention teaches cultured iPSC cells on top of collagen-sandwiched hepatocytes. The uniform microenvironment, three-dimensional collagen gel, and cues from isolated hepatocytes contributed to the formation of a homogeneous cell population.

As used herein, the term “osteoblast differentiation agent” refers to one of a collection of compounds that are used in culture systems of this invention to produce differentiated cells of the mesenchymal lineage (including pre-osteoblasts, osteoblasts and terminally differentiated cells). No limitation is intended to be placed on the mode of action of the osteoblast differentiation agents. For example, the agent may facilitate the differentiation process by inducing or assisting a change in phenotype, promoting growth of cells with a particular phenotype or retarding the growth of others, or acting in concert with other agents through unknown mechanisms.

In some situations canine MSC are utilized to generate bone in vitro or in vivo, general examples of suitable osteoblast differentiation agents may include but are not limited to one or more of the following general compounds: (1) bone morphogenic proteins, exemplified by BMP-2, BMP-3, BMP-4, BMP-6 and BMP-7; (2) TGF-β, exemplified by TGF-β1, TGF-β2, and TGF-β3 and their analogs, and other members of the TGF-β superfamily that bind a TGF-β receptor; and (3) ligands for the Vitamin D receptor. Exemplary is 1.25-dihydroxyvitamin D3. Other known vitamin D3 analogs may also be suitable (see, for example, Tsugawa et al., (2000) Biol. Pharm. Bull. 23:66). Most preferably, however, the osteoblast differentiation agents of the invention include the following non-limiting compounds and their respective concentration ranges: 1.25(OH)2D3 (10-9 to 10-7M), dexamethasone (10-9 to 10-7M), retinoic acid (10-9 to 10-5M), or combinations thereof, in addition to ascorbic acid (50 to 100 μg/ml) and β-glycerophosphate (1 to 10 mM). These agents are supplied to the mesenchyme-specific culture medium in a sequence-specific manner, by adding an effective amount of 1.25(OH)2D3, dexamethasone, retinoic acid or combinations thereof to the medium followed by the addition of ascorbic acid and β-glycerophosphate to promote differentiation of the MSCs into a substantially homogenous population of bone precursor cells. These organic compounds are generally used to mimic local bone microenvironments. Applicants note that with the knowledge of the specific concentrations of osteoblast differentiation agents provided in the example below, those skilled in the art would be able to readily determine what would constitute an “effective amount” or optimal concentration range for osteoblast differentiation agents.

Furthermore, it is recognized that antibodies specific to the receptors of any of these factors are functionally equivalent ligands that can be used in place of (or in addition to) the factors listed. Other additives that may be used include: other morphogens, such as a fibroblast growth factor like basic FGF; a glucocorticoid; dexamethasone, or other small-molecule osteoblast maturation factor; ascorbic acid (or an analog thereof, such as ascorbic acid-2-phosphate), which is a cofactor for proline hydroxylation that occurs during the course of collagen synthesis; β-glycerophosphate, or other substrate for alkaline phosphatase during the process of mineralization

It is understood by the skilled artisan that bone precursor cells will typically have at least one characteristic and typically at least three or five of the following characteristics: 1) density between ^{˜}1.050 and ^{˜}1.090 g cm-3; 2) positive for osteonectin (positive in osteoblasts and precursors) and related osteoblast transcription factors described below; 3) positive for osteocalcin (specific for mature osteoblasts); 4) a cell diameter of ^{˜}8 to ^{˜}70 micron; 5) cuboidal shape; 6) upregulated production of alkaline phosphatase, especially in response to presence of BMP; 7) positive for type I collagen (procollagen) or for vimentin; 8) positive for other osteoblast-specific markers, such as BMP receptors, PTH receptors, or CD105 (endoglin); 9) evidence of ability to mineralize the external surroundings, or synthesize calcium-containing extracellular matrix. To determine the function of the homogenous population of bone precursor cells, expression of tissue-specific gene products may be detected at the mRNA level by Northern blot analysis, dot-blot hybridization analysis, or by reverse transcriptase initiated polymerase chain reaction (RT-PCR) using sequence-specific primers in standard amplification methods. See for example: U.S. Pat. No. 5,843,780 for details of general technique, and International Patent Publication WO 99/39724 for osteoblast-specific PCR primers. Sequence data for other markers listed in this disclosure can be obtained from public databases such as GenBank (URL www.ncbi.nlm.nih.gov:80/entrez). Expression at the mRNA level is generally “detectable” according to any one of the assays described in this disclosure providing the performance of the assay on cell samples results in clearly discernable hybridization or amplification product. Expression of tissue-specific markers as detected at the protein or mRNA level is considered positive if the level is at least 2-fold, and preferably more than 10- or 50-fold above that of a control cell, such as an undifferentiated MSC population or other unrelated cell type.

1. A canine-derived mesenchymal stem cell derived from an induced pluripotent stem cell (iPSC).

2. The canine-derived mesenchymal stem cell of claim 1, capable of more than 20 doublings without losing therapeutic activity by more than 10%.

3. The canine-derived mesenchymal stem cell of claim 1, wherein said induced pluripotent stem cells are cultured under conditions conducive to form embryoid bodies (EBs), wherein subsequently said EBs are exposed to a mesenchyme-specific medium containing 1.25(OH)2D3 to promote formation of cells of the mesenchymal lineage.

4. The canine-derived mesenchymal stem cell of claim 1, wherein said stem cell is capable of suppressing T cell proliferation.

5. The canine-derived mesenchymal stem cell of claim 2, wherein said stem cell is capable of enhancing T regulatory cell proliferation.

6. The canine-derived mesenchymal stem cell of claim 2, wherein said stem cell is capable of enhancing angiogenesis.

7. The therapeutic activity of claim 2 is defined by the ability of conditioned media from the mesenchymal stem cell to stimulate proliferation of canine-derived endothelial cells by more than 25% as compared to baseline.

8. The canine derived mesenchymal stem cell of claim 1, wherein said induced pluripotent stem cells are cultured under conditions conducive to form embryoid bodies (EBs), wherein subsequently said EBs are exposed to a mesenchyme-specific medium containing 1.25(OH)2D3 to promote formation of EBs having cells of the mesenchymal lineage.

9. The mesenchyme-specific media of claim 8 contains VEGF.

10. The canine-derived mesenchymal stem cell of claim 9, wherein the mesenchyme-specific media contains canine monocyte conditioned media.

11. The conditioned media of claim 10 is generated by culturing canine peripheral blood mononuclear cells in an adherent vessel, washing off non-adherent cells, culturing monocytes in liquid media, and collecting the liquid media as conditioned media.

12. The mesenchymal stem cell of claim 1 expresses the Cba-1 gene.

13. The mesenchymal stem cell of claim 1 expresses higher levels of hTERT compared to a mesenchymal stem cell isolated from canine tissue.

14. A method of treating canine hepatic failure comprising:
a) obtaining canine induced pluripotent stem cells (iPSCs);
b) creating embryoid bodies from said iPSCs;
c) isolating mesenchymal progenitor cells from said embryoid bodies;
d) expanding mesenchymal stem cell populations from said embryoid bodies;
e) priming said mesenchymal stem cells towards hepatic differentiation; and
f) administering said hepatic-primed mesenchymal stem cells to a canine in need of therapy in an amount sufficient to treat said hepatic failure.

15. The method of claim 14, wherein histone deacetylase inhibitors or hypoxia conditions are employed during transfection.

16. The method of claim 15, wherein said hypoxia conditions induce HIF-1α translocation by more than 10%, 25%, or 100%.

17. The method of claim 15, wherein said hypoxia conditions are combined with a cellular stress selected from the group consisting of hyperthermia, hypertonic, and hypotonic stress.

18. The method of claim 14, wherein hepatic-primed MSCs are pretreated with a molecule to enhance homing to the hepatic microenvironment, selected from the group consisting of HGF and CXCR4.

19. The method of claim 1, wherein exosomes are derived from cells selected from the group consisting of: iPSCs, MSCs, and hepatocyte progenitors.

20. The method of claim 14, wherein said exosomes are characterized by having a size between 50-300 nm and a marker selected from the group consisting of CD63, miR-155, and CD8.
